(19) United States
(12) Patent Application Publication
(10) Pub. No.: US 2006/0111577 A1
(43) Pub. Date: May 25, 2006

(54) Method for preparing 17 alpha-acetoxy-11beta-(4-n,n-dimethylaminophenyl)-19-norpregna-4,9-diene-3,20-dione, intermediates thereof, and methods for the preparation of such intermediates

(76) Inventors: Hyun K. Kim, Bethesda, MD (US); Pemmaraju Narasimha Rao, San Antonio, TX (US); Anne Marie Simmons, San Antonio, TX (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6780, US

(21) Appl. No.: 10/542,580

(22) PCT Filed: Feb. 13, 2004
(86) PCT No.: PCT/US04/04246

§ 371 Date: Nov. 28, 2005

(30) Foreign Application Priority Data
Feb. 28, 2003 (US) 60451096

(51) Int. Cl. C07J 5/00; C07J 7/00

(52) U.S. Cl. 552/557

(57) ABSTRACT

Methods for the preparation of the 19-norprogesterone of formula I and its intermediates, in crystalline and amorphous forms.

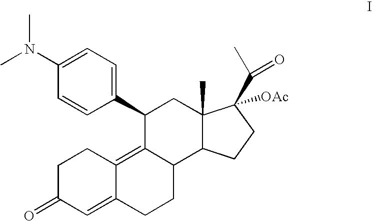

### CROSS-REFERENCE TO RELATED APPLICATION

This application claims the benefit of U.S. provisional patent application No. 60/451,096, filed Feb. 28, 2003, the disclosure of which is incorporated by reference.

### FIELD OF THE INVENTION

The present invention relates generally to steroids and, in particular, to methods for the preparation of 17α-acetoxy-11β-(4-N,N-dimethylaminophenyl)-19-norpregna-4,9-diene-3,20-dione, intermediates useful in those methods, and methods for the preparation of such intermediates.

### BACKGROUND OF THE INVENTION

The compound 17α-acetoxy-11β-(4-N,N-dimethylamino-phenyl)-19-norpregna-4,9-diene-3,20-dione, represented by formula I, 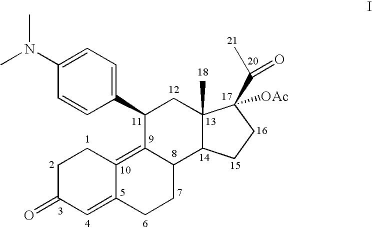
is a well-known steroid, more specifically a 19-norprogesterone, which possesses antiprogestational and antiglucocorticoidal activity. This compound, and methods for its preparation, are described in U.S. Pat. Nos. 4,954,490, 5,073,548, and 5,929,262 (“the '262 patent”).

A method for the preparation of the 19-norprogesterone described in the '262 is reproduced in FIG. 1. This method begins by converting the dienone, 17α-hydroxy-19-norpregna-4,9-diene-3,20-dione II, to a bis-ketal compound A via a reaction with ethylene glycol and triethylorthoformate in the presence of an acid catalyst. The bis-ketal compound A is then epoxidized using hexafluoroacetone/H₂O₂ in the presence of sodium phosphate dibasic to provide the epoxide compound of formula B. The epoxide then undergoes conjugate ring-opening using a copper (I)-catalyzed Grignard reagent generated by the reaction of 4-bromo-N,N-dimethylaniline with magnesium in the presence of copper (I) to provide compound C. A hydrolysis/dehydration procedure is then used to convert compound C to the compound D, which is acetylated to produce the desired 19-norprogesterone of formula I (indicated as compound I in FIG. 1).

While the foregoing procedure can be used to prepare the 19-norprogesterone of formula I, certain drawbacks are inherent in the procedure. More specifically, the foregoing procedure includes processing steps, which are not readily amenable to the preparation of commercial quantities of the desired 19-norprogesterone. The method described in the '262 patent, for example, requires the formation of a bis-ketal compound which does not proceed to completion (only 60% yield at best) and involves extensive chromatographic separation in order to purify the bis-ketal product. Deprotection of the bis-ketal is also not quantitative. As a result of these shortcomings, the overall yield provided by this known process is relatively low.

In view of the foregoing, a need exists for a more efficient process for the preparation of the 19-norprogesterone of formula I and intermediates thereof, in particular a process that results in the production of these compounds in relatively large and highly pure quantities. It is an object of the present invention to provide such a method. This and other objects and advantages of the invention, as well as additional inventive features, will be apparent from the description of the invention provided herein.

### BRIEF SUMMARY OF THE INVENTION

The invention provides a method for the preparation of the 19-norprogesterone of formula I and its intermediates. The method is more efficient than currently available methods, providing such compounds in relatively large and highly pure quantities.

With respect to the preparation of the 19-norprogesterone of formula I, the present invention comprises acetylating the hydroxyl group in the compound of formula II 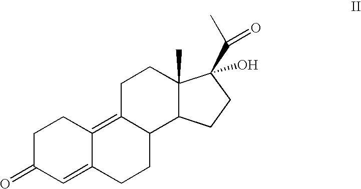
to provide the compound of formula III, 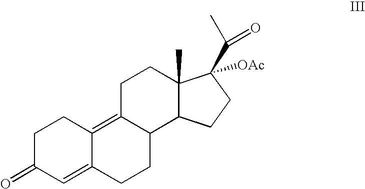
ketalizing the 3-carbonyl group of the compound of formula III to provide the compound of formula IV, 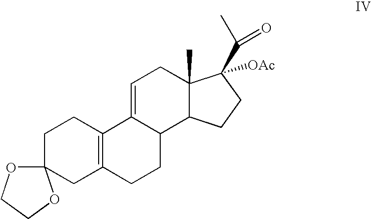
epoxidizing the compound of formula IV to provide the 5α, 10α-epoxide compound of formula V, 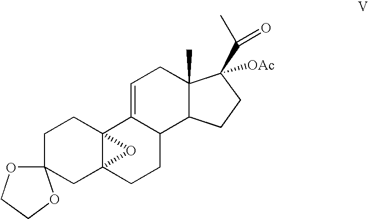
reacting the compound of formula V with a N,N-dimethylaminophenyl reactant to provide the compound of formula VI, and 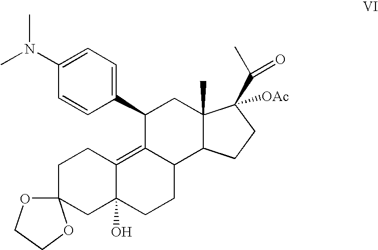
deketalizing and dehydrating the compound of formula VI to provide the compound of formula I. By following the foregoing method, one is able to obtain the desired 19-norprogesterone in a relatively high yield and at a high purity level. As mentioned previously, another aspect of the present invention provides methods for the preparation of several of the intermediates useful for the preparation of the 19-norprogesterone of formula I, e.g., intermediates II, III, IV, V, and VI, as well as methods of converting one intermediate to another.

### BRIEF DESCRIPTION OF THE DRAWINGS

FIG. 1 sets forth a known method for the preparation of the 19-norprogesterone of formula I.

FIG. 2 sets forth a method for the preparation of the 19-norprogesterone of formula I in accordance with an embodiment of the present invention.

### DETAILED DESCRIPTION OF THE INVENTION

The present invention provides a method for preparing the compound of formula I (i.e., 17α-acetoxy-11β-(4-N,N-dimethylaminophenyl)-19-norpregna-4,9-diene-3,20-dione). 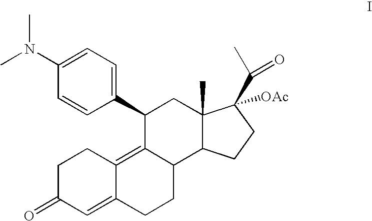
Preferably, the starting material used in the method of the present invention is a compound of formula II (i.e., 17α-hydroxy-19-norpregna-4,9-diene-3,20-dione). 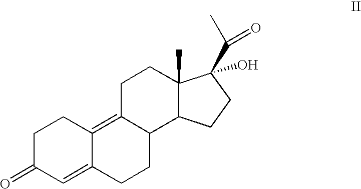
This compound can be obtained by previously known synthetic methods, such as the method described in the '262 patent.

In accordance with the present invention, the 17-hydroxyl group in the compound of formula II is protected. Preferably, the 17-hydroxyl group is acetylated to form a compound of formula III (i.e., 17α-acetoxy-19-norpregna-4,9-diene-3,20-dione).

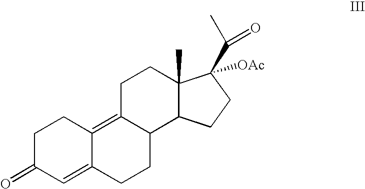

Any suitable acetylating agents can be utilized. For example, acetic acid, acetylchloride, anhydrides (e.g., acetic anhydride), or a mixed anhydride of acetic acid, combinations thereof, and the like can be used. Advantageously, a mixed anhydride procedure employing a trifluoroacetic anhydride/acetic acid mixture is used. p-Toluene sulfonic acid can be used as a catalyst.

Desirably, the molar amount of trifluoroacetic anhydride is approximately equal to, or greater than, the molar amount of the acetic acid and the molar amount of the compound of formula II. Preferably, the molar amounts of the trifluoroacetic anhydride and acetic acid are up to about 20 times or more than the molar amount of the compound of formula II.

Examples of solvents suitable for the acetylation reaction include, but are not limited to, dichloromethane, tetrahydrofuran (THF), diethyl ether, acetonitrile, dioxane, and the like, with dichloromethane being a preferred solvent. During the reaction, the reactants are advantageously maintained at a temperature of from about −10° C. to about 30° C., and most preferably at a temperature of about 0° C.

When the acetylation reaction is complete, the reaction mixture is cooled and neutralized via dropwise addition of base (e.g., ammonium hydroxide, sodium carbonate, sodium bicarbonate, or another suitable base). Preferably, the mixture is neutralized with an ammonium hydroxide solution. The mixture is then diluted with water and extracted with an organic solvent (e.g., dichloromethane). Compound of formula III can be obtained by crystallization from the extract solution in 62% yield.

After the compound of formula III is prepared, the 3-carbonyl group of that compound is ketalized to provide the compound of formula IV (i.e., 3,3-ethylenedioxy-17α-acetoxy-19-norpregna-5(10),9(11)-diene-20-one). 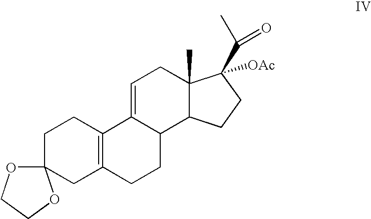
The ketalization step can be conducted in any suitable manner, but is preferably undertaken by reacting the compound of formula III with a diol in the presence of an acid.

Any suitable acid may be used in the foregoing reaction to catalyze the formation of the ketal. Suitable acids for this purpose include organic and inorganic acids. Preferably, an organic acid is used to catalyze ketal formation. When an organic acid is used, it is preferably selected from sulfur-based organic acids, e.g., methanesulfonic acid, ethanesulfonic acid, benzenesulfonic acid, and naphthalenesulfonic acid, with toluenesulfonic acid being the most preferred.

Any suitable diol may be used for the formation of the ketal. The diol can be provided in excess with respect to the carbonyl group(s) being ketalized, so as to favor the formation of the ketal. Preferably, the diol used in the ketalization step is ethylene glycol.

Suitable water scavengers can be used in the foregoing reaction to remove chemically the water from the ketalization reaction and drive the reaction to completion. Suitable water scavengers include, for example, orthoesters, particularly orthoformate esters, which are advantageous in that they provide high yields. Preferred orthoformate esters include triisobutyl orthoformate, triisopropyl orthoformate, and triethyl orthoformate, with triethyl orthoformate being most preferred.

The ketalization reaction preferably is conducted in the presence of a solvent, which is preferably a halogenated solvent. Suitable halogenated solvents include chloroform, dichloromethane, dichloroethane, and trichloroethane, with a preferred solvent being dichloromethane.

Compound of formula IV can be purified using any suitable purification method, but is preferably purified by crystallization from ethyl acetate. Quantitative yields of compound of formula IV have been obtained by recrystallization of the ketalization product from boiling ethyl acetate. The high yield for mono-ketalization is in marked contrast to the yield of 60% or less that was achieved for bis-ketalization following the method of the '262 patent. In addition, isolation of pure mono-ketal intermediate can be achieved efficiently without the need for laborious chromatography.

The compound of formula IV is then epoxidized to form the 9,11-unsaturated 5α, 10-epoxide of formula V (i.e., 3,3-ethylenedioxy-5α,10α-epoxy-17α-acetoxy-19-norpregna-9(11)-ene-20-one). 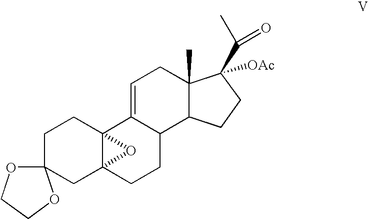
The epoxidation reaction can be carried out using any suitable epoxidation procedure, but is preferably accomplished by reacting the compound of formula IV with a halogenated acetone and a peroxide in the presence of an inorganic base. Any suitable peroxide, or peracid, can be used in this reaction. Examples of suitable peroxides include hydrogen peroxide, sodium peroxide, potassium peroxide, benzoyl peroxide, and acetyl peroxide, with the preferred peroxide being aqueous hydrogen peroxide, most preferably 30 wt. % hydrogen peroxide in water.

The halogenated acetone can be any suitable acetone that provides the desired results. Preferably, a hexahalogenated acetone is used, e.g., hexafluoroacetone, hexachloroacetone, or hexabromoacetone, with hexafluoroacetone being preferred.

The reaction is preferably carried out in the presence of an inorganic base, which is most preferably a phosphate base or a carbonate (or bicarbonate) base. Examples of suitable phosphate bases include di- and tri-basic sodium and potassium phosphate. Suitable carbonate bases include sodium and potassium carbonate, and sodium and potassium bicarbonate. Most preferably, the epoxidation reaction is carried out in the presence of dibasic sodium phosphate. Especially preferred is the use of dibasic sodium phosphate in combination with the 30 wt. % hydrogen peroxide and hexafluoroacetone.

The epoxidation reaction is further advantageously conducted in the presence of a solvent, which is preferably a halogenated solvent. Suitable halogenated solvents include chloroform, dichloromethane, dichloroethane, and trichloroethane, with the most preferred solvent being dichloromethane.

The compound of formula V can be crystallized using an ether, e.g., diethyl ether, isopropyl ether, isobutyl ether, and n-butyl ether, with diethyl ether being preferred.

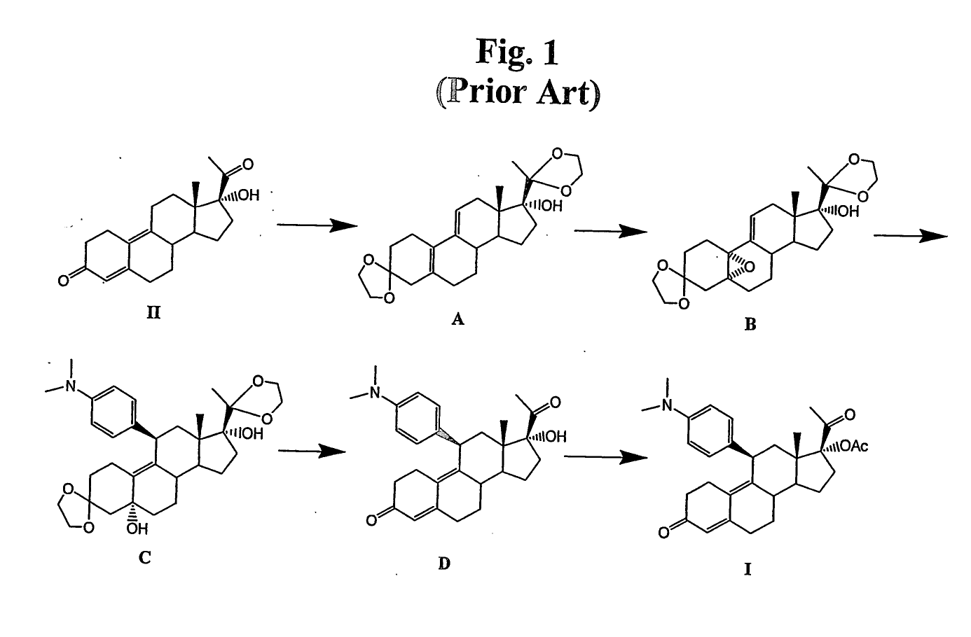

The stereoselectivity of the 5α,10α-epoxide versus the 5β10β-epoxide was 7:1 using the 17α-acetoxy intermediate (compound IV) in accordance with the present invention. By contrast, a 3:1 mixture of the 5α,10α-epoxide and 5β,10β-epoxide was obtained using the bis-ketal (compound A of FIG. 1) according to the method of the '262 patent. Because of the advantageously high α-epoxide to β-epoxide ratio obtained in accordance with the present invention, isolation of the desired 5α,10α-epoxide product via chromatography was not necessary.

After forming the ketal protecting group, the epoxide in the compound of formula VI undergoes a conjugate ring-opening reaction, and a N,N-dimethylaminophenyl functional group may be substituted, preferably in the axial position, of C₁₁, to provide the compound of formula VI (i.e., 3,3-ethylenedioxy-5α-hydroxy-17α-acetoxy-11β-4-(N,N-dimethylaminophenyl)-19-norpregna-9-ene-20-one). 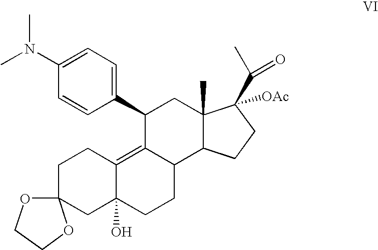
The foregoing reaction preferably is performed by reacting the compound of formula V with a Grignard reagent prepared from the reaction of p-bromo-N,N-dimethylaniline and magnesium in the presence of a cuprous halide (e.g., cuprous chloride). The reaction is further advantageously conducted in the presence of a solvent. For example, the solvent can be dry THF or an ether, such as diethyl ether, with a preferred solvent being dry THF.

It was surprisingly discovered that, when this reaction scheme was undertaken, less Grignard reagent was required as compared to the amount of Grignard reagent required in the conversion of the 17α-hydroxy epoxide material (epoxide B of FIG. 1) disclosed in the '262 patent. For example, the reaction may be carried out with about a two-fold excess of Grignard reagent relative to the epoxide in accordance with the present invention. By contrast, nearly five-fold excess of Grignard reagent is used in the process described in the '262 patent.

Compound of formula VI is further advantageously obtained in crystalline form by crystallization from an ether, preferably, diethyl ether.

The compound of formula VI is then deketalized and dehydrated to provide the compound of formula I. The foregoing conversion of the compound of formula VI to the compound of formula I preferably is performed by reaction with an acid. The acid can serve the dual function of hydrolyzing the ketal group (i.e., deketalization) and removing the hydroxyl at C₅ position (i.e., dehydration). Any suitable acid that functions to hydrolyze the ketal group can be used in accordance with the present invention. Suitable acids include, for example, acetic acid, sulfuric acid, hydrochloric acid, and phosphoric acid. Preferably, the acid is acetic acid.

The deketalization reaction is preferably conducted in the presence of a solvent. The solvent can be any suitable solvent, for example THF, diethyl ether, acetonitrile, dioxane, dichloromethane, and the like, with a preferred solvent being THF. The deketalization reaction advantageously can be carried out under reflux conditions.

After its formation, the compound of formula I can be crystallized from acetone/hexane in high yield (e.g., 82% yield) and in high purity.

The present inventive method for preparing the compound of formula IV from the compound of formula II was surprisingly found to provide a greater yield than known methods requiring bis-ketalization. Although not wishing to be bound by any particular theory, it is believed that the C20 ketone group is sterically hindered by the presence of the 17α-acetoxy group, thus rendering it substantially unreactive toward other chemical reagents. This discovery led to the elimination of the low yield bis-ketalization step used in conventional methods.

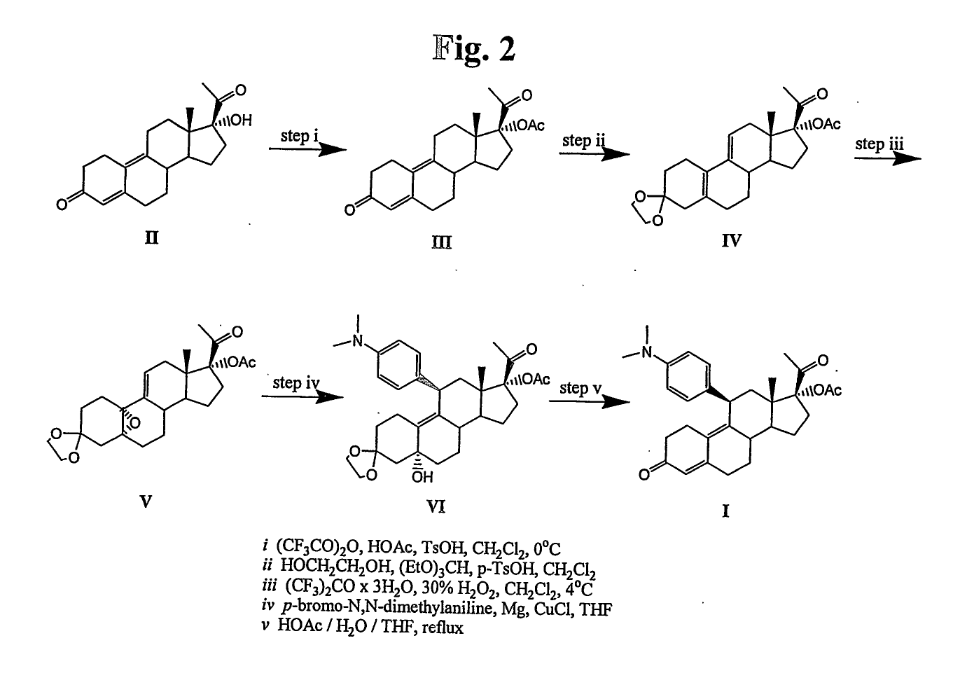

From an overall perspective, the present inventive method provides a much greater yield of the final product of formula I as compared to yields obtained using conventional methods, and also avoids the need for laborious chromatographic separation, making the present inventive method more readily amenable to scale-up. By following the methods of the present invention, one may obtain an overall yield of the compound of formula I of about 20% or more starting from compound of formula II. This is contrasted with known methods, such as the method described in the '262 patent which provides an overall yield of about 12%. A preferred embodiment of the present inventive reaction scheme is depicted in FIG. 2.

The present invention further allows one to prepare any of the intermediates described herein starting from the compound of formula II, or any other preceding intermediate, as well as the compound of formula I starting from any of the aforesaid.

The following examples further illustrate the invention but, of course, should not be construed as in any way limiting its scope.

### EXAMPLE 1

The Preparation of the Compound of Formula II (17α-Hydroxy-19-Norpregna-4,9-diene-3,20-dione) from 3,20-bis-ethylenedioxy-17α-hydroxy-19-norpregna-5(10),9(11)-diene.

Under nitrogen, 3,20-bis-ethylenedioxy-17α-hydroxy-19-norpregna-5(10),9(11)-diene (20 g, 49.8 mmol) in tetrahydrofuran (333 mL) was treated with water (333 mL) followed by trifluoroacetic acid (1 L, 13.46 mol). The reaction mixture was stirred at room temperature for 2 hr., after which time, TLC (10% acetone in CH₂Cl₂; overspotted with concentrated NH₄OH) indicated a complete reaction. The reaction mixture was cooled in an ice bath and neutralized by the dropwise addition of concentrated (29.5%) ammonium hydroxide solution (862 mL, 13.46 mol) over a period of about an hour. The reaction mixture was diluted with water (500 mL) and extracted with CH₂Cl₂ (3×). The organic fractions were washed with saturated sodium bicarbonate solution (1×), water (1×), and brine (1×), then filtered through anhydrous sodium sulfate, combined and concentrated in vacuo. Crystallization of the residue from acetone/hexanes gave 12.0 g of the purified product (II) in two crops as a pale yellow solid in 77% yield; m.p. 203-205° C.

FTIR (KBr, diffuse reflectance): νₘₐₓ 3438, 2950, 1702, 1642, and 1593 cm⁻¹. NMR (300 MHz, CDCl₃) δ 0.857 (s, 3H, C18-CH₃), 2.289 (s, 3H, C21-CH₃), and 5.669 (s, 1H, C4-CH═) ppm. MS (E1) m/z (relative intensity): 314 (M+, 100), 296 (14), 271 (58), 213 (67), and 91 (36). Anal. Calcd. for C₂₀H₂₆O₃: C, 76.40; H, 8.34. Found: C, 76.23; H, 8.29.

### EXAMPLE 2

The Preparation of the Compound of Formula III (17α-Acetoxy-19-Norpregna-4,9-diene-3,20-dione) from the Compound of Formula II (17α-Hydroxy-19-Norpregna-4,9-diene-3,20-dione).

A mixture of trifluoroacetic anhydride (67 mL, 487 mmol) and glacial acetic acid (28 mL, 474 mmol) in CH₂Cl₂ (420 mL) was stirred for ½ hr at room temperature under nitrogen, then cooled to 0° C. in an ice bath. Toluenesulfonic acid (4.0 g, 21.0 mmol) was added as a solid, followed by a solution of the 17-hydroxy steroid II (6.0 g, 19.1 mmol) in CH₂Cl₂ (60 mL). The steroid II was rinsed in with an additional CH₂Cl₂ (60 mL). After ¼ hr. at 0° C., TLC (2% acetone in CH₂Cl₂) indicated all starting material had been converted to one major product. The reaction mixture was diluted with H₂O (30 mL) and quenched at 0° C. by the careful addition of concentrated NH₄OH (97 ml). Additional NH₄OH was added until the pH was approximately 7. The mixture was transferred to a separatory funnel and the layers allowed to separate. The organic phase was washed with H₂O (2×). Combined CH₂Cl₂ extracts (3×) were dried by filtration through Na₂SO₄ and evaporated in vacuo. The resulting residue was dried overnight to afford 7.27 g of a yellow foam. Trituration of this foam with pentane produced a yellow powder, which was collected on a Buchner funnel and washed with ether. The material was dried overnight to afford 1.85 g of highly purified product as evidenced by TLC (5% acetone in CH₂Cl₂). A second crop (1.19 g) of equal purity was obtained by crystallization of the mother liquors from hot ether. Flash chromatography (5% acetone in CH₂Cl₂) of the mother liquors followed by crystallization from hot ether gave 1.2 g of additional product. The total yield of purified material (III) was 4.24 g in 62.3% yield; m.p. softens at 119° C.

FTIR (KBr, diffuse reflectance): νₘₐₓ 2941, 1733, 1716, 1653, and 1600 cm⁻¹. NMR (300 MHz, CDCl₃) δ 0.796 (s, 3H, C18-CH₃), 2.072 and 2.116 (2s, 6H, C17-OC(O)CH₃ and C21-CH₃), and 5.708 (s, 1H, C4-CH═) ppm. MS (E1) m/z (relative intensity): 356 (M⁺, 5.9), 296 (18.4), 271 (10.9), and 253 (100.0). Anal. Calcd. for C₂₂H₂₈O₄: C, 73.20; H, 7.96. Found: C, 73.06; H, 7.89.

### EXAMPLE 3

The Preparation of the Compound of Formula IV (3,3-Ethylenedioxy-17α-Acetoxy-19-Norpregna-5(10),9(11)-diene-20-one) from the Compound of Formula III (17α-Acetoxy-19-Norpregna-4,9-diene-3,20-dione).

To a solution of the 17α-acetoxy derivative (III) (4.24 g, 11.8 mmol) in CH₂Cl₂ (60 mL) was added triethylorthoformate (4.96 mL, 29.2 mmol) and ethylene glycol (3.46 mL, 61.8 mmol). The mixture was stirred at room temperature, under nitrogen, as toluenesulfonic acid (114 mg, 0.6 mmol) was added as a solid. After ½ hr., TLC analysis (5% acetone in CH₂Cl₂) indicated all of the starting material had been converted to a single, less polar product. The reaction mixture was transferred to a separatory funnel and washed with saturated NaHCO₃ (1×), H₂O (1×), and brine (1×). Combined CH₂Cl₂ extracts (3×) were dried by filtration through Na₂SO₄ and evaporated in vacuo. The resulting pale yellow solid was dried further under high vacuum to afford 4.82 g of IV in quantitative yield. A small amount was crystallized from boiling EtOAc containing a few drops of CH₂Cl₂ for characterization; m.p. softens at 232° C.

FTIR (KBr, diffuse reflectance): νₘₐₓ 2903, 1728, 1712, 1444, 1368, and 1255 cm⁻¹. NMR (300 MHz, CDCl₃) δ 0.618 (s, 3H, C18-CH₃), 2.066 and 2.091 (2s, 6H, C17-OC(O)CH₃ and C21-CH₃), 3.986 (s, 4H, C3-OCH₂CH₂O), and 5.597 (s, 1H, C11-CH═) ppm. MS (EI) nz (relative intensity): 400 (M+, 6.2), 340 (15.3), 297 (100.0), and 211 (53.1). Anal. Calcd. for C₂₄H₃₂O₅: C, 71.97; H, 8.05. Found: C, 72.14; H, 8.11.

### EXAMPLE 4

The Preparation of the Compound of Formula V (3,3-Ethylenedioxy-5α, 10α-epoxy-17α-acetoxy-19-norpregna-9(11)-en-20-one) from the Compound of Formula IV (3,3-Ethylenedioxy-17α-Acetoxy-19-Norpregna-5(10),9(11)-diene-20-one).

Solid Na₂HPO₄ (1.16 g, 8.2 mmol) was added to a solution of hexafluoroacetone trihydrate (1.71 mL, 12.3 mmol) in CH₂Cl₂ (35 mL), followed by 30% H₂O₂ (1.85 mL, 18 mmol). The mixture was stirred vigorously for ½ hr. at 4° C. in the cold room. A chilled solution of the 3-ketal (IV) (3.28 g, 8.2 mmol) in CH₂Cl₂ was added and rinsed in with additional CH₂Cl₂ (10 mL). The reaction was stirred overnight at 4° C. The next morning, TLC (5% acetone in CH₂Cl₂) indicated all starting material had been converted to one major, more polar product. A trace of several more polar impurities appeared. The reaction mixture was transferred to a separatory funnel and washed with 10% Na₂SO₄ and evaporated in vacuo to afford 3.73 g of a pale yellow foam. Trituration with ether produced 2.71 g of a white solid (V) in three crops in 80% yield. Examination by TLC indicated all of the three crops were highly purified. NMR showed a mixture of α:β in a ratio of 87:13; m.p. softens at 139-152° C.

FTIR (KBr, diffuse reflectance): νₘₐₓ 2926, 1722, 1441, 1371, and 1260 cm⁻¹. NMR (300 MHz, CDCl₃) δ 0.620 (s, 3H, C18-CH₃), 2.064 and 2.079 (2s, 6H, C17-OC(O)CH₃ and C21-CH₃), 3.928 (s, 4H, C3-OCH₂CH₂O), 5.864 (m, 0.13H, C11β—CH═), and 6.056 (m, 0.87H, C11α—CH═) ppm. MS (EI) m/z (relative intensity): 416 (M+, 2.6), 398 (9.7), 356 (45.7), 313 (100.0), and 99 (98.0). Anal. Calcd. for C₂₄H₃₂O₆ ½H₂O: C, 68.84; H, 7.76. Found: C, 68.94; H, 7.81.

### EXAMPLE 5

The Preparation of the Compound of Formula VI (3,3-Ethylenedioxy-5α-hydroxy-17α-Acetoxy-11β-4-(N,N-dimethylaminophenyl)-19-Norpregna-9-en-20-one) from the Compound of Formula V (3,3-Ethylenedioxy-5α,10α-epoxy-17α-acetoxy-19-norpregna-9(11)-en-20-one).

A dry 250 mL round bottom flask was equipped with a reflux condenser, a stirring bar and rubber septum. Magnesium (342 mg, 14.1 mmol), was added and the entire assembly was dried with a heat gun under a stream of nitrogen. The apparatus was allowed to cool slightly and one crystal of iodine was added. After cooling completely, dry THF (13 mL; Na/benzophenone) was added, followed by one drop of 1,2-dibromoethane. A solution of 4-bromo-N,N-dimethylaniline (2.56 g, 12.8 mmol) in dry THF (7 mL) was added via transfer needle and rinsed in with an additional 6 mL of THF. The mixture was gently heated to reflux with a heat gun to initiate reaction, then allowed to stir 1 hr. at room temperature. Copper (I) chloride (140 mg, 1.4 mmol) was added as a solid and stirring continued for ½ hr. A solution of the 5α,10α-epoxide (V) (2.66 g, 6.4 mmol, 87% a) in THF (20 mL) was added via transfer needle, then rinsed with 6 mL of additional THF. After stirring 2 hr at ambient temperature the reaction was quenched by the addition of saturated NH₄Cl (52 mL). Air was drawn through the mixture for 12 hr. with vigorous stirring. The mixture was transferred to a separatory funnel, H₂O and ether were added, and the layers allowed to separate. The organic fraction was washed again with H₂O, and then brine. Combined ether extracts (3×) were dried by filtration through Na₂SO₄ and evaporated in vacuo to afford a blue-brown foam (3.95 g). Ether was added and a small amount of a fine brown precipitate formed. This was removed by filtration through a sintered glass funnel and the filtrate was concentrated to a syrup. The addition of a seed crystal, with scratching, produced a mass of crystals. The slurry was transferred to a centrifuge tube and the crystals collected by centrifugation. After washing with several small portions of ether, TLC indicated the product was highly purified. The product was dried overnight under vacuum to afford 1.43 g of a pale brown powder in 47.8% yield, based on 87% of the starting material being α-epoxide; m.p. 220-223° C.

FTIR (KBr, diffuse reflectance): νₘₐₓ, 3528, 2937, 1731, 1714, 1612, and 1518 cm⁻¹. NMR (300 MHz, CDCl₃) δ 0.286 (s, 3H, C18-CH₃), 2.067 and 2.108 (2s, 6H, C17-OC(O)CH₃ and C21-CH₃), 2.898 (s, 6H, —N(CH₃)₂), 3.992 (m, 4H, C3-OCH₂CH₂O), 4.285 (d, 1H, C11α—CH—, J=7.8 Hz), 6.623 (d, 2H, 3′,5′-aromatic CH, J=8.7 Hz), and 7.015 (d, 2H, 2′,6′-aromatic CH, J=8.7 Hz) ppm. MS (EI) m/z (relative intensity): 537 (M⁺, 46.6), 519 (15.3), 134(19.8), 121 (100.0), and 99 (10.7). Anal. Calcd. for C₃₂H₄₃O₆N: C, 71.48; H, 8.06, N, 2.60. Found: C, 71.65; H, 8.27; N, 2.73.

### EXAMPLE 6

The Preparation of the Compound of Formula I from the Compound of Formula VI (3,3-Ethylenedioxy-5α-hydroxy-17α-Acetoxy-11β-4-(N,N-dimethylaminophenyl)-19-Norpregna-9-en-20-one).

To a solution of the Grignard product VI (2.09 g, 3.9 mmol) in THF (25 mL) was added glacial acetic acid (75 mL) followed by H₂O (25 mL), and the mixture brought to reflux under nitrogen. After 2.5 hr., the reaction was cooled to room temperature, then placed in an ice water bath and neutralized by the careful addition of concentrated NH₄OH (88 mL). Additional NH₄OH was added until the pH reached approximately 7. The mixture was transferred to a separatory funnel, CH₂Cl₂ was added, and the layers allowed to separate. The organic fraction was washed with H₂O, and then brine. Combined CH₂Cl₂ extracts (3×) were dried by filtration through Na₂SO₄ and evaporated in vacuo to afford 1.95 g of a brown foam. The crude material was taken up in hot EtOH and treated with activated charcoal. The mixture was filtered through a Celite® filter cake while still hot and washed with several portions of hot EtOH. Evaporation of the filtrate gave a yellow glass. Crystallization of the yellow glass from acetone/hexane gave 1.57 g of I as yellow crystals in three crops in 82.0% yield. For purpose of analysis, a portion of this material was further purified by flash chromatography and recrystallizations from aqueous ethanol (90%) resulting in large white crystals with characteristics identical to those reported for previously prepared material.

HPLC Analysis on a Water Associate's NovaPak C18 column, eluded with 70% CH₃OH in water with 0.05% triethylamine at a flow rate of 1 mL/min and at λ=260 nm indicated a purity of 99.0% with a retention time (t_{R}) of 7.24 min. The product co-eluted with an authentic sample of previously prepared material; m.p.=183-185° C.

FTIR (KBr, diffuse reflectance): νₘₐₓ 2966, 2944, 2880, 2840, 2796, 1730, 1717, 1661, 1611, 1596, 1574, 1515, and 810 cm⁻¹. NMR (300 MHz, CDCl₃)δ 0.360 (s, 3H, C18-CH₃), 2.094 and 2.132 (2s, 6H, C17-OC(O)CH₃ and C21-CH₃), 2.907 (s, 6H, —N(CH₃)₂), 4.386 (d, 1H, C11α—CH—, J=7.2 Hz), 5.775 (br s, 1H, C4—CH═—)6.636 (d, 2H, 3′,5′-aromatic CH, J=8.85 Hz), and 6.978 (d, 2H, 2′,6′-aromatic CH, J=8.85 Hz) ppm.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for preparing the compound of formula I, 17α-acetoxy-11β-(4-N,N-dimethylaminophenyl)-19-norpregna-4,9-diene-3,20-dione, 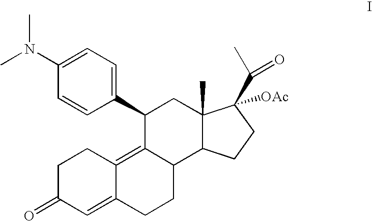
comprising:
(i) acetylating the 17α-hydroxyl group of a compound of formula II 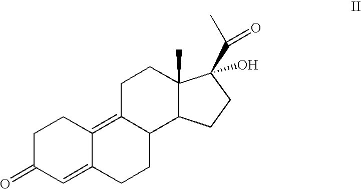
to produce a compound of formula III; 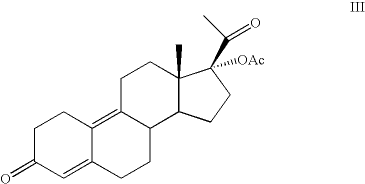
(ii) ketalizing the 3-keto group of the compound of formula III to produce a compound of formula IV; 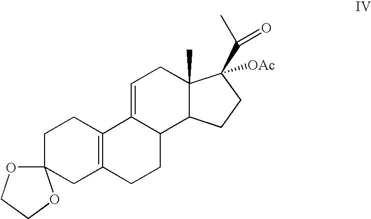
(iii) epoxidizing the compound of formula IV to produce a compound of formula V; 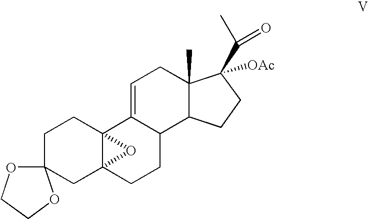
(iv) reacting the compound of formula V with a N,N-dimethylaminophenyl reactant to produce a compound of formula VI; 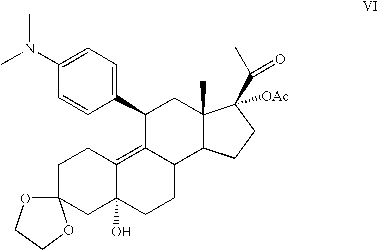
and
(v) deketalizing and dehydrating the compound of formula VI.

2. A method for preparing a compound of formula IV 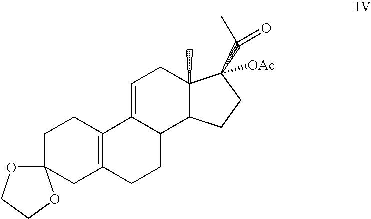
comprising:
(i) acetylating the 17α-hydroxyl group of the compound of formula II 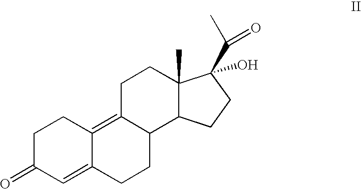
to produce a compound of formula III; 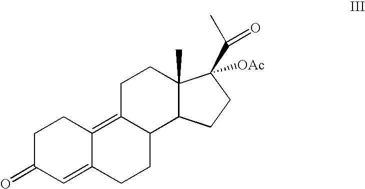
and
(ii) ketalizing the 3-keto group of the compound of formula III.

3. A method for preparing a compound of formula V 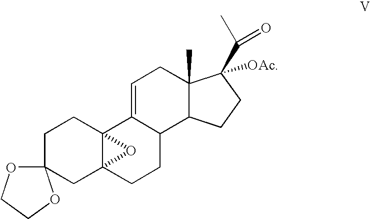
comprising reacting the compound of formula IV with a N,N-dimethylaminophenyl reactant comprising a Grignard reagent of the formula (Me)₂NC₆H₄MgX, wherein X is a halogen, and a cuprous halide.

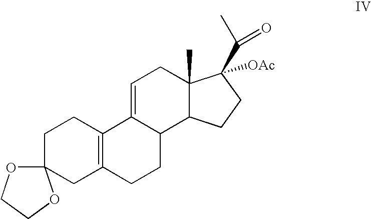

4. The method of claim 3, wherein the amount of cuprous halide is less than an equimolar amount relative to the epoxide.

5. The method of claim 3, wherein the N,N-dimethylaminophenyl reactant is prepared by the reaction of p-bromo-N,N-dimethylaniline and magnesium in the presence of a cuprous chloride.

6. The method of claim 3, wherein the Grignard reagent is provided in a molar amount which is about two times or less the molar amount relative to the epoxide.

7. A method for preparing a compound of formula I 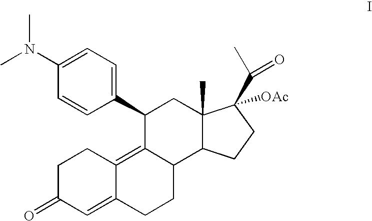
comprising deketalizing and dehydrating the compound of formula VI,

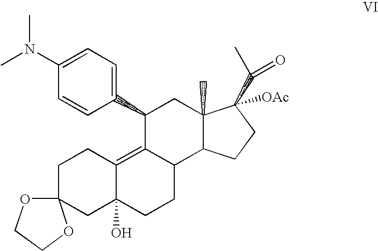

8. The method of claim 7, further comprising purifying compound of formula I to produce a crystalline form which has a melting point from about 183° C. to about 185° C.

9. The method of claim 1, wherein the acetylating step is performed by reacting the compound of formula II with a mixture prepared from trifluoroacetic anhydride, acetic acid, and p-toluenesulfonic acid.

10. The method of claim 9, wherein the molar amount of trifluoroacetic anhydride is approximately equal to the molar amount of the acetic acid and the molar amount of the compound of formula II.

11. The method of claim 10, wherein the molar amounts of the trifluoroacetic anhydride and acetic acid are up to about 20 times or more than the molar amounts of the compound of formula II.

12. The method of claim 2, wherein the acetylating step is performed by reacting the compound of formula II with a mixture prepared from trifluoroacetic anhydride, acetic acid, and p-toluenesulfonic acid.

13. The method of claim 12, wherein the molar amount of trifluoroacetic anhydride is approximately equal to the molar amount of the acetic acid and the molar amount of the compound of formula II.

14. The method of claim 12, wherein the molar amounts of the trifluoroacetic anhydride and acetic acid are up to about 20 times or more than the molar amount of the compound of formula II.

15. The method of claim 7, wherein compound of formula VI is prepared by reacting the compound of formula V, 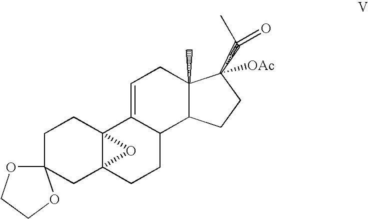
with a N,N-dimethylaminophenyl reactant.

16. The method of claim 15, wherein compound of formula V is prepared by epoxidizing the compound of formula IV,

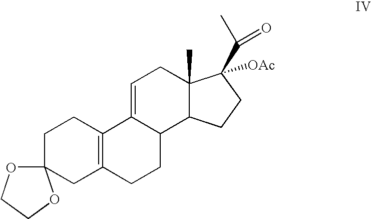

17. The method of claim 16, wherein the compound of formula IV is prepared by ketalizing the 3-keto group of the compound of formula III,

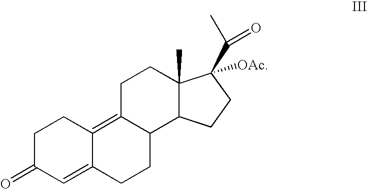

18. The method of claim 17, wherein compound of formula III is prepared by acetylating 17α-hydroxy-19-norpregna-4,9-diene-3,20-dione (compound of formula II)

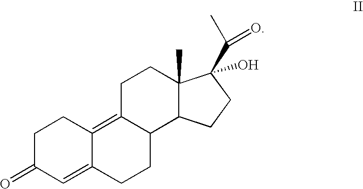

19. A method for preparing compound of formula III comprising acetylating 17α-hydroxy-19-norpregna-4,9-diene-3,20-dione (compound II)

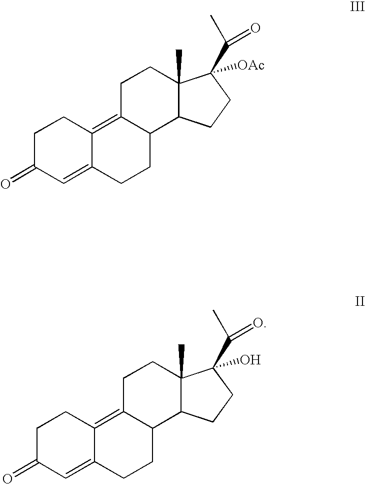

20. A method for preparing compound of formula IV 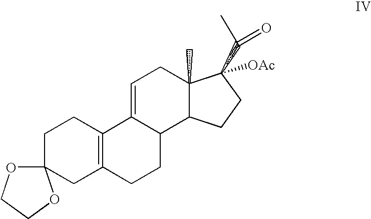
comprising ketalizing the 3-keto group of the compound of formula III

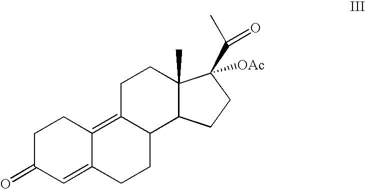

21. A method for preparing compound of formula VI 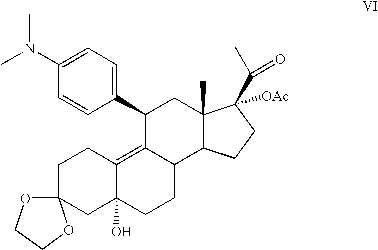
comprising reacting the compound of formula V 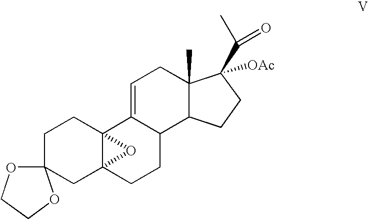
with a N,N-dimethylaminophenyl reactant.

22. A compound of formula III (17α-acetoxy-19-norpregna-4,9-diene-3,20-dione),

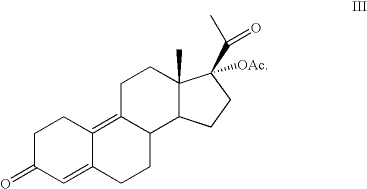

23. A compound of formula IV (3,3-ethylenedioxy-17α-acetoxy-19-norpregna-5(10),9(11)-diene-20-one),

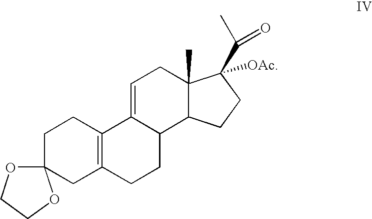

24. A compound of formula V (3,3-ethylenedioxy-5α,10α-epoxy-17α-acetoxy-19-norpregna-9(11)-ene-20-one),

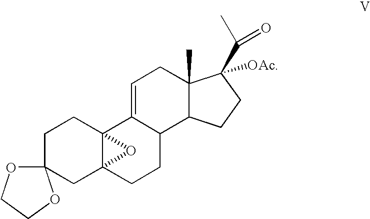

25. A compound of formula VI (3,3-ethylenedioxy-5α-hydroxy-17α-acetoxy-11β-4-(N,N-dimethylaminophenyl)-19-norpregna-9-ene-20-one), [chemical structure]
